(19) United States
(12) Patent Application Publication
(10) Pub. No.: US 2023/0390503 A1
(43) Pub. Date: Dec. 7, 2023

(54) DELIVERY DEVICE AND COATED NEEDLE OR CANNULA

(71) Applicant: Becton, Dickinson and Company, Franklin Lakes, NJ (US)

(72) Inventors: Christopher MAUL, Belton, TX (US); Christopher GILDEA, Sparta, NJ (US); Andrew METTERS, Westford, MA (US)

(73) Assignee: Becton, Dickinson and Company, Franklin Lakes, NJ (US)

(21) Appl. No.: 18/248,031

(22) PCT Filed: Oct. 4, 2021
(86) PCT No.: PCT/US2021/053309

§ 371 Date: Apr. 5, 2023

Related U.S. Application Data
(60) Provisional application No. 63/089,420, filed on Oct. 8, 2020.

(51) Int. Cl. A61M 5/32

(52) U.S. Cl. CPC A61M 5/3286; A61M 2205/0238

(57) ABSTRACT

A medication delivery device is provided with a stainless steel needle or cannula where the patient end of the needle or cannula has a protective outer layer over the surface of the needle or cannula. The protective outer layer has a thickness and is oriented to prevent or minimize contact of the tissue of the patient with the stainless steel to prevent or inhibit allergic reactions by the tissue contact with the nickel in the stainless steel. The protective outer layer can be a metal coating of a non-allergenic metal, such as gold, or other coating material that is able to cover the exposed surface of the needle to prevent direct contact of the stainless steel with the patient.

### RELATED APPLICATIONS

This application is a National Stage Patent Application based on PCT Patent Application No. PCT/US2021/053309, filed Oct. 4, 2021, which claims priority to U.S. Provisional Patent Application Ser. No. 63/089,420, filed Oct. 8, 2020. Each of these applications is hereby incorporated by reference in its entirety.

### FIELD OF THE INVENTION

The present invention is directed to a delivery device and a cannula or needle for a delivery device having a coating to inhibit allergic reactions when in contact with tissue of a patient. The cannula or needle can be made of stainless steel and have an outer layer or coating of a non-allergenic material to prevent or minimize direct contact of the stainless steel with the patient during use.

### DESCRIPTION OF THE RELATED ART

Various injection devices have been produced that include a conventional cannula or needle for introducing a medicament intravenously, intramuscular or subcutaneously. The cannula or needle is commonly made of stainless steel. Some patients exhibit an allergic reaction to the nickel in the stainless steel cannula or needle. In particular, long term exposure of the stainless steel in contact with the skin or other tissue of the patient can lead to allergic reactions and discomfort to the patient.

An example of common delivery device is a syringe or pen-injector delivery device to facilitate self-administration of parenteral medications. Pen needles are a component of needle-based injection systems and consist of a doubled ended cannula assembled into a hub using an adhesive. The hub has internal threads, which allow it to be attached to the pen-injector device. Pen needle attachment allows the proximal end of cannula to penetrate through the rubber septum of the medicament cartridge in the delivery device to create the fluid flow path. For many diabetics maintaining blood glucose control is achieved by performing multiple daily injections of insulin into the subcutaneous (SC) tissue using pen injector delivery devices developed to be a convenient, discreet alternative to the vial and syringe. Numerous pen injectors are commercially available in either disposable or multi-use configurations, each offering various patient-centric features. The distal pen needle cannula interfaces with the delivery site providing a conduit for delivery. Pen needle designs are intended to enable consistent delivery to the target tissue space, minimize leakage of medication, and reduce pain/discomfort and site effects such as bleeding and bruising associated with the injection. The primary design features, needle length/gauge and hub face geometry, in conjunction with mechanics of the delivery system and injection technique, dictate injection success.

Injections may be performed in the intradermal region, the subcutaneous region and the intramuscular (IM) region of the skin. For many types of injectable medications, including insulin, the SC region is preferred for administering an injection. See, for example, Lo Presti, et al., Skin and subcutaneous thickness at injecting sites in children with diabetes: ultrasound findings and recommendations for giving injection, Pediatric Diabetes (2012).

Needle lengths, such as needles having a length of about 4 mm to 5 mm are adapted to inject a medication to a specified target depth in a subcutaneous region. The present delivery device provides a structure so that a needle can be consistently inserted to a desired target depth. Prior pen needles have the cannula supported on an axial post extending from the hub. A post forms a narrow portion and a relatively wider base that does not contact the skin during the injection. In other pen needles known in the art, a distal face of the hub placed against the injection site may be relatively large, and may be provided with a slight taper at the edge. The edge of the hub can engage the skin when the cannula is inserted at an angle relative to the surface of the skin of the patient.

While the prior devices are generally suitable for the intended use, there is a continuing need for improved devices for delivering a drug or medicament to a selected target area.

### SUMMARY OF THE INVENTION

The present invention is directed to a delivery device, such as an injection device having a stainless steel component that is coated with a protective layer to prevent or inhibit direct contact of the stainless steel with the subject. In one embodiment, the delivery device is a syringe or needle hub for use with an injection device such as a pen-injector. In other embodiments, the delivery device can be an infusion set or patch pump for providing a continuous or sustained delivery of a medication.

The delivery device as disclosed includes a stainless steel component. In the embodiments described, the stainless steel component is a cannula or needle having a coating or outer layer made of a non-allergenic material to prevent or inhibit allergic reactions with the metal or compounds in the cannula or needle. The cannula or needle is typically made of a suitable stainless steel for surgical purposes. The coating or protective layer covers the stainless steel to avoid or minimize contact with the tissue of a patient during use.

The coating or protective layer of the cannula or needle can be any suitable material that can adhere to the cannula or needle during use and inhibit or reduce the surface contact of the stainless steel with the tissue of the patient while the cannula or needle is in use and in contact with the tissue of the patient. The coating in one embodiment is a metal coating that does not induce allergic reactions when in contact with the tissue of a patient and will effectively prevent or reduce direct contact of the stainless steel with the tissue during use. The metal coating can be a noble metal, such as gold. Other metals can include silver, palladium, ruthenium, osmium and rhenium.

The delivery device in one embedment can be any suitable delivery device that utilizes a cannula or needle for delivering a medication to a patient. The delivery device can be a syringe having a needle extending from a distal end of the syringe. In other embodiments, the delivery device can be an infusion set having a flexible cannula or catheter, patch pump having flexible cannula or catheter, or an IV catheter where the delivery device has an insertion or introducer needle for introducing the delivery device into the patient.

In one embodiment the delivery device is an injection device configured for injecting a drug, medicament, or other substance to a patient at a desired depth relative to the surface of the skin. The delivery device can be a needle hub having a hub face forming a contact surface with the skin where the hub face has a configuration for controlling and optimizing the depth of penetration by a needle or cannula. The needle hub can be optimized to determine the depth of insertion of the cannula or needle such that the entire length of the exposed cannula or needle is inserted into the tissue.

The delivery device in one embodiment is a syringe having a needle having a sharpened tip extending from a distal end for penetrating the skin of the patient and delivering a medication. The needle can be made of stainless steel. At least a portion of the outer surface of the needle can be covered or coated with a material forming a protective layer. The protective layer does not cause an allergic reaction with the skin and isolates the stainless steel needle from the patient to prevent contact of the stainless steel needle with the tissue and inhibit or reduce an allergic reaction by the patient.

The coating on the needle can cover at least a portion of the length of the needle. In one embodiment, the coating covers the entire or substantially the entire length of the exposed portion of the needle that is intended to come into contact with the tissue of the patient. In other embodiments, the coating can cover a distal end portion that is configured to contact the tissue of the patient. In another embodiment, the coating can cover only a portion of the needle spaced from the distal end of the needle so that the coated portion contacts the tissue of the patient to prevent contact of the tissue with the stainless steel.

The needle in another embodiment can be an insertion needle or guide wire for a catheter or flexible cannula of an infusion set. In this embodiment, the distal end of the needle extending from the catheter or flexible cannula can be covered by a protective coating while the portion of the needle within the catheter or flexible cannula that does not come into contact with the skin is not covered by the protective coating.

The features of the device include a delivery device for delivering a substance, such as a medication, to a patient. The delivery device in one embodiment includes a needle made of stainless steel where at least a portion of the needle is covered by a protective coating to prevent contact of the stainless steel with the tissue of the patient.

A method of preventing or inhibiting an allergic reaction by the patient is provided by preventing direct contact of the stainless steel component of the delivery device with the skin or tissue of the patient. The method contacts the delivery device with the skin or tissue of the patient and where the delivery device provides a coating of a non-allergenic material on the portion of the delivery in contact with the patient to prevent or inhibit an allergic reaction by the patient caused by stainless steel.

It will be understood that each of the preferred or optional features of the various embodiments may be combined with other features and features described in combination with one or more particular features may also be combined with one or more other features of the other embodiments.

These and other features of the invention will become apparent from the following detailed description of the invention, which in conjunction with the drawings disclose various embodiments of the invention.

### BRIEF DESCRIPTION OF THE DRAWINGS

The following is a brief description of the drawings in which:

FIG. 1 is an elevational view of a syringe and needle in one embodiment;

FIG. 2 is an elevational view of a needle in one embodiment having a coating of a non-allergenic material;

FIG. 3 is an elevational view of the needle having a coating of a non-allergenic material on a distal end portion;

FIG. 4 is an elevational view of the needle having a coating of a non-allergenic material on a portion of the needle spaced from the distal tip;

FIG. 5 is a side view in partial cross section showing the needle inserted into a lumen;

FIG. 6 is a side view of a catheter and catheter insertion needle;

FIG. 7 is a side view in cross section of a pen needle hub; and

FIG. 8 is side view in cross section of a delivery device having a flexible cannula and introducer needle.

### DETAILED DESCRIPTION OF EXEMPLARY EMBODIMENTS

The delivery device as described can be a suitable device having a stainless steel component that typically contacts and penetrates the skin during use. An example of a delivery device is a pen needle or syringe. The pen needle refers to a needle hub attached to a pen-injector device for injecting a medication or other substance into a patient. The terms needle and cannula refer to a thin tubular member for insertion into an injection site on a subject having a lumen for delivering the substance to the patient. The needle typically has a sharpened end for piercing the skin of the patient. A distal direction is in the direction toward the injection site, and the proximal direction is the opposite direction. The axial direction refers to a direction along or parallel to the longitudinal axis of the needle and the needle hub and the radial direction refers to a direction perpendicular to the axial direction. The pen-injector device can be a standard device as known in the art where the needle hub is attached to the end of the pen-injector for delivering the substance to a patient. After use, the needle hub is removed and discarded and replaced with a new needle hub for a subsequent injection. The features of the different embodiments can be combined and used interchangeably where the embodiments are not inconsistent with one another.

The delivery device as described and illustrated has at least one stainless steel component that is protected from direct contact with the tissue of patient by a protective layer or coating to prevent direct contact of the stainless steel component with the tissue or skin of the patient. The stainless steel can be a surgical grade stainless steel, such as stainless steel type 304 having a minimum chromium content of 18% and a minimum nickel content of 8% by weight. Stainless steel has a nickel content that can cause an allergic response when in contact with the skin of a subject that is allergic to nickel. Stainless steel needles that are inserted into the skin or tissue of a patient can increase the allergic reaction at the injection site. The allergic reactions to nickel can result in the formation of a rash or bumps on the skin, itching, redness, changes in color, dry patches or blisters. In some instances, the allergic reaction can be severe.

The invention is directed to medication delivery device, such as an injection device having a stainless steel component, such as a needle or cannula that is covered by a protective layer to inhibit such allergic reactions. The stainless steel cannula or needle can have a chromium content of about 10.5% to about 35%. In one embodiment, the cannula or needle can have a chromium content of about 11% to 24%, and typically about 11.5% to about 14.5%. The needle can a length and gauge suitable for the intended injection. Suitable examples of needle length for a syringe can be 1 inch up to 2 inches, and commonly 1½ inch. The gauge of the needle is also selected based on the intended use and can range from 16 gauge to 36 gauge. An insertion or introducer needle can be, for example, 16 to 18 gauge. A needle for subcutaneous in injection can be ½ to ⅝ inch long and a gauge of 25-30. In one embodiment, the needle or cannula can be for example, 4-8 mm and can be 31-36 gauge. In other embodiments, the needle or cannula can be other gauges as suitable for the intended purpose.

The delivery device can be any suitable device for delivering a medication to a patient that uses a needle or cannula to deliver the medication or introducing a catheter into the patient. Examples of delivery devices can be syringes, catheters, reusable or disposable injector devices, autoinjectors, syringes, patch-pumps or other delivery devices.

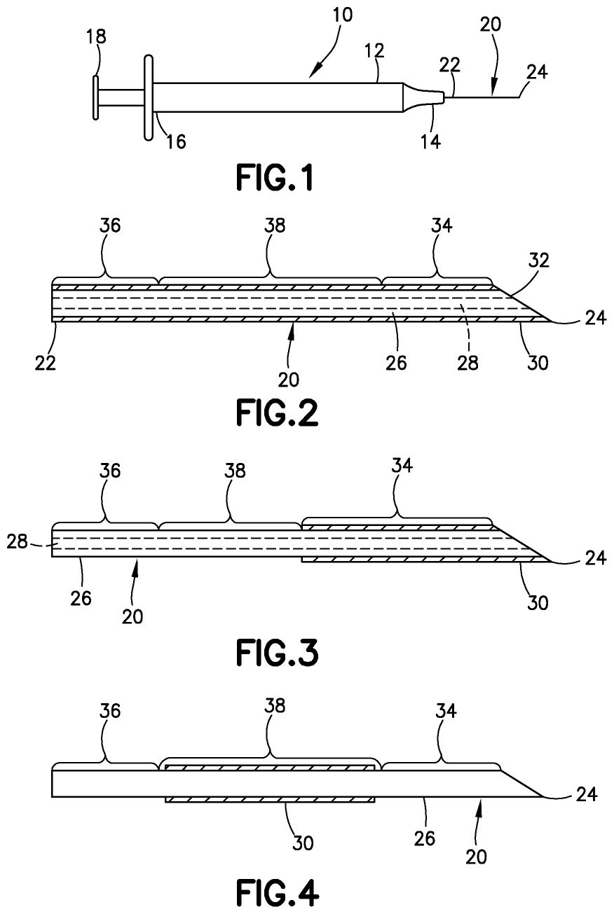

In a first embodiment shown in FIG. 1, the delivery device is syringe 10 having a syringe barrel 12, a distal tip 14, and a proximal end 16. A plunger 18 is provided to dispense the contents of the syringe in a usual manner. A needle 20 extends from the distal tip 14 and has an outer surface that is exposed for penetrating the skin of the patient.

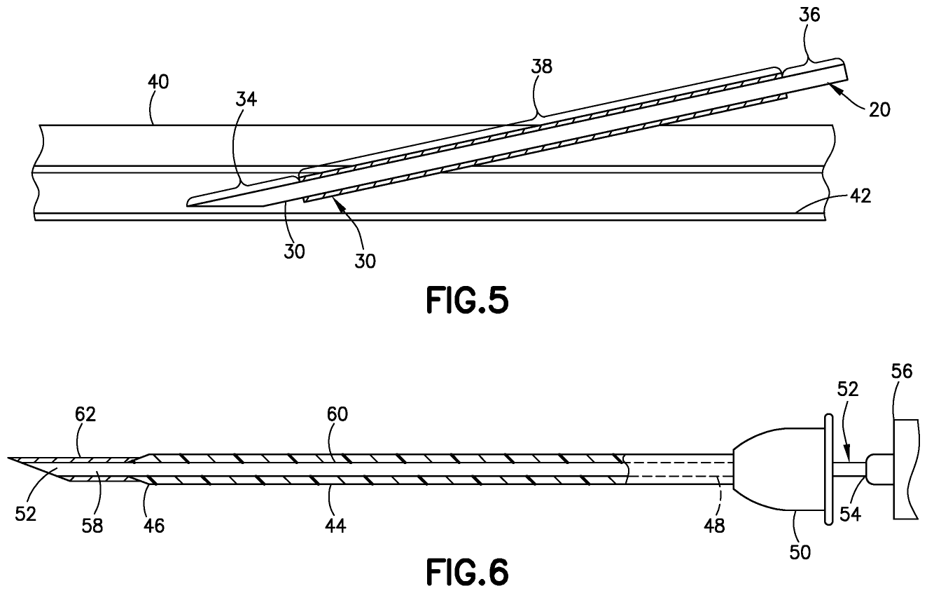

The needle 20 as shown in the embodiment of FIG. 2 has a proximal end 22 for coupling to the delivery device, such as the syringe 10, a sharpened distal tip 24, and a body 26 extending between the distal tip 22 and the proximal end 22. In the embodiment shown, a lumen 28 extends axially through the needle body 26 for delivering the medication from the delivery device to the patient. The needle is typically made of stainless steel, such as a surgical grade stainless steel. At least a portion of the outer surface of the needle body 26 has a protective layer or coating 30 to enclose or encapsulate the surface of the stainless steel to prevent contact of the outer surface of the needle with the patient. The protective layer is made of a material that is non-allergenic so that contact with the skin or tissue of the patient does not cause an allergic reaction and protects the patient from directly contacting the stainless steel.

The protective layer 30 in one embodiment is a metal coating that is applied directly on the outer surface of the stainless steel needle. The protective layer has a thickness and orientation to prevent or minimize contact of the stainless steel with the patient during use. The metal is typically a noble metal. A particularly suitable metal protective layer for stainless steel is gold that can be applied by various coating techniques, such as a standard gold electroplating process. Gold also provides a visual indicator for the patient to identify a delivery device with the protective layer. Other metals that can provide a suitable protective layer include platinum, iridium, silver, rhenium, ruthenium, rhodium, palladium, iridium, and osmium. In other embodiments, the protective layer can be anodized, ceramic material or a polymer coating. The polymer coating can also provide a lubricating effect to assist in inserting the needle into the patient. The outer surface of the protective layer can include a conventional lubricant used for needles and cannulas. Examples of a lubricant can be a silicone oil or a silicone polymer or resin coating fixed to the outer surface of the stainless steel.

As shown in FIG. 2, the protective layer is formed on the outer surface of the needle body 26 and covers substantially the entire surface extending from the proximal end 22 to the distal tip. In one embodiment, the protective layer can be formed on the bevel 32 of the needle 20.

The needle body 26 of the needle 20 has a distal end portion 34 with an outer surface, a proximal end portion 36 with an outer surface, and an intermediate surface 38 between the distal end portion 34 and the proximal end portion 36. In the embodiment of FIG. 2, the outer protective layer covers the distal end portion 34, the proximal end portion 36, and the intermediate surface 38.

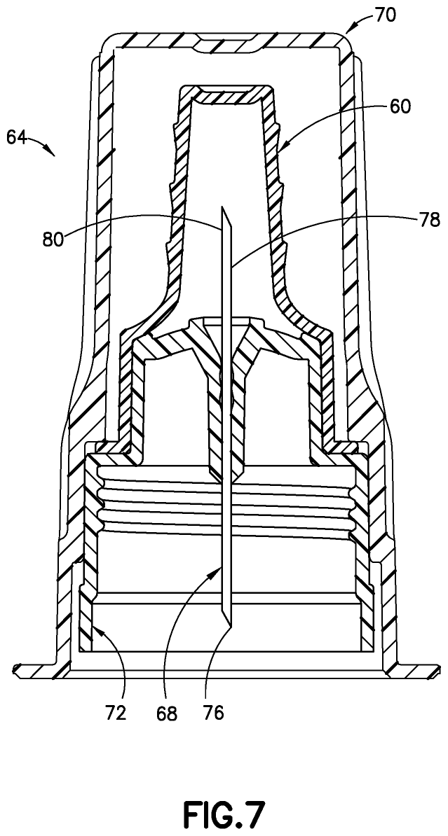

In another embodiment shown in FIG. 3, the protective coating 30 is formed only on the distal end portion 36. The intermediate surface 38 and the proximal end portion 36 in the embodiment shown are not intended to contact the subject so these portions are not covered by the protective layer. In the embodiment shown in FIG. 3, the needle 20 is configured so that the distal end portion is positioned in the tissue or skin of the patient, while the intermediate surface and proximal end portion are not covered by the protective layer 30. The protective layer 30 in this embodiment covers a portion of the needle that is typically in contact with the skin or tissue during use. As in the previous embodiment, the protective layer can be a gold coating applied directly on the surface of the stainless steel needle.

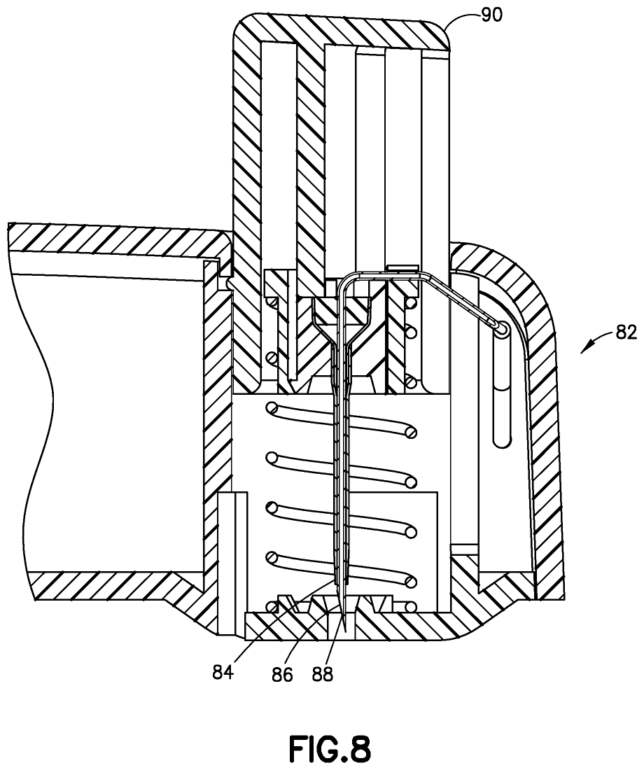

In an embodiment as shown in FIG. 4, the needle 20 has a portion of the needle body 26 covered with the protective layer to overlie the stainless steel surface. As shown the protective layer is applied to an intermediate portion 30 spaced from the distal end portion 34. In the embodiment shown, the protective layer 30 is also spaced from the proximal end 36 of the needle.

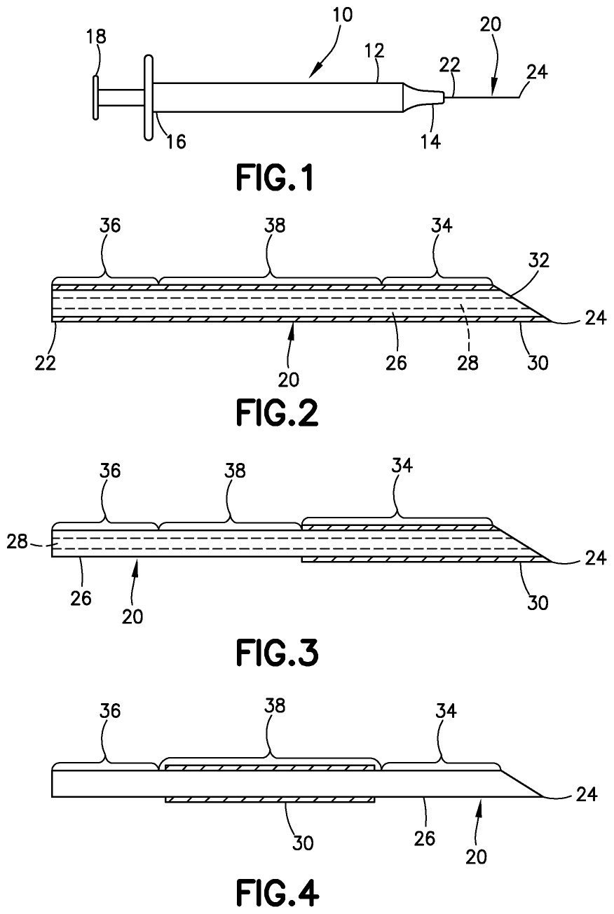

The needle 20 of FIG. 4 can be an intravenous (IV) needle configured for positioning in the vein or artery of the patient. The IV needle can be for introducing a substance into the patient or can a blood draw needle. As shown in FIG. 5 the needle is inserted through the skin 40 and into a vein 42 where the distal tip 14 and the distal end portion 34 are positioned within the lumen 44 of the vein 42 and do not directly contact the tissue. The intermediate surface 38 covered by the protective layer 30 is oriented on the needle so that only the intermediate surface portion and the protective layer directly contact the tissue or skin at the insertion site to inhibit the allergic response by the nickel contained in the stainless steel.

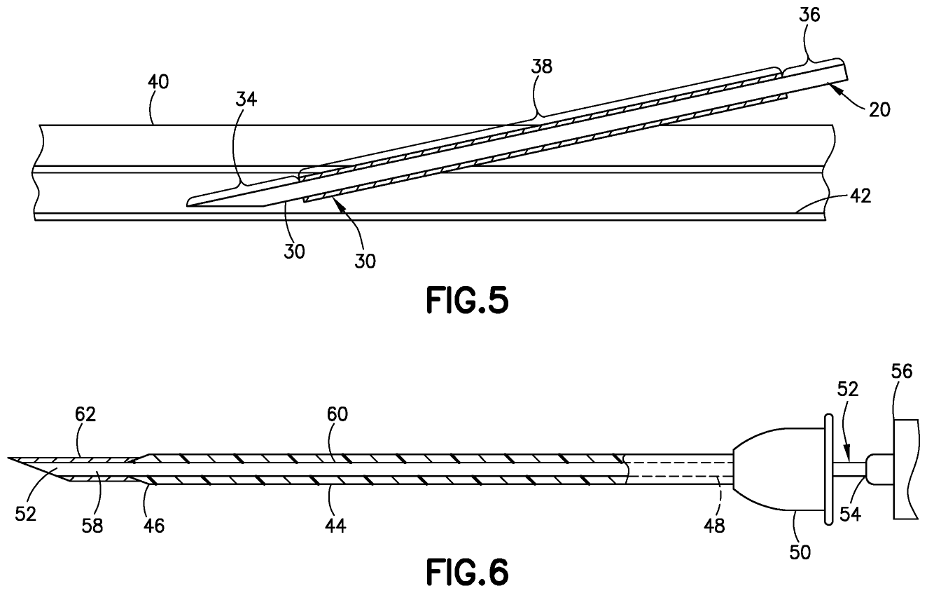

In another embodiment shown in FIG. 6, the delivery device is a catheter 44 having a lumen, a distal end 46, a proximal end 48, and a catheter hub 50 coupled to the distal end of the catheter 44. The catheter is typically a flexible catheter made from a polymeric material for introducing a medication into a patient. An introducer needle 52 is initially oriented in the lumen of the catheter 44 and extends from the distal end of the catheter for introducing the flexible catheter in the selected location. Once the catheter is positioned in the selected location, the needle can be removed from the catheter so that the catheter is able to introduce a medication to the patient. The introducer needle 52 has a proximal end 54 coupled to a needle retractor mechanism 56 to retract the introducer needle once the catheter is positioned in the patient. The introducer needle is made of stainless steel as in the previous embodiments. Typically the introducer needle has a lumen and a sharpened distal tip as shown. The introducer needle 52 has a distal end portion 58 extending from the distal end of the catheter 44 and an intermediate portion 60 spaced from the distal end portion 58 and positioned within the catheter so that only the distal end portion is exposed. In the embodiment shown, the distal end portion 58 of the introducer needle 52 has a protective layer 62 forming a coating to cover the surfaces of the stainless steel introducer needle.

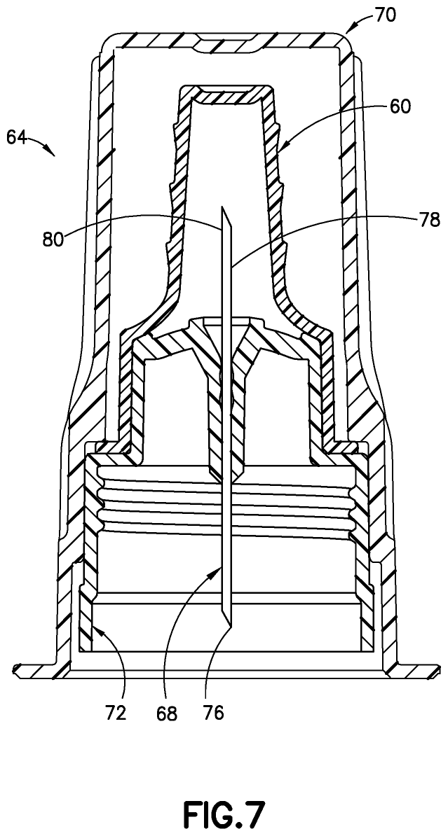

In a further embodiment shown in FIG. 7, the delivery device is a pen needle 64. The pen needle 64 in the embodiment shown has an inner shield 66 to cover the needle 68 and an outer cover 70. The pen needle 64 has needle hub 72 with an open proximal end 74 for coupling with a delivery pen. The needle 68 in the embodiment shown has a length to extend through the needle hub 72 and has a sharpened proximal end 76 for piercing a septum of the delivery pen. As shown in FIG. 7, the needle 68 has an exposed distal portion 78 extending from the needle hub for penetrating the skin of the patient to deliver the medication. As in the previous embodiment, the exposed portion 78 of the needle 68 is covered by a protective layer 80 of a non-allergenic material, such as a gold coating. The coating of the protective layer on the exposed distal portion of the needle prevents direct contact of the stainless steel needle with the tissue of the patient during the delivery of the medication to prevent or inhibit an allergic reaction by the nickel in the stainless steel.

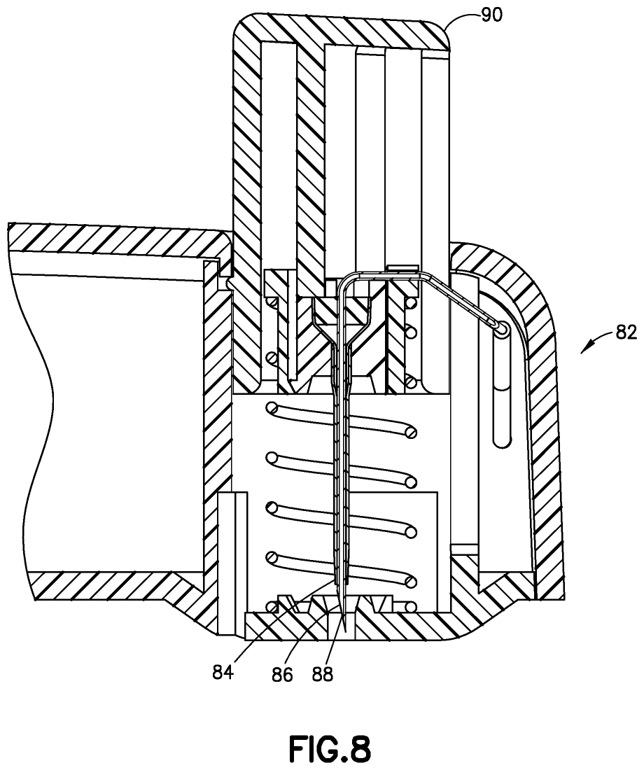

In another embodiment shown in FIG. 8, the delivery device 82 is a patch pump or infusion device. The patch pump 82 and a soft, flexible catheter 84 connected to fluid source for delivering the medication to the patient. An insertion needle 86 is initially positioned in the lumen of the catheter 84 with the distal end 88 extending from the distal end of the catheter. The patch pump includes an insertion actuator 90 for inserting the insertion needle and catheter into the patient and then retracts the insertion needle so that the catheter can deliver the medication. In the embodiment shown, the distal end 88 of the insertion needle 86 is provided with the protective layer to prevent contact of the tissue of the patient with the stainless steel insertion needle during insertion to inhibit an allergic reaction by the nickel contained in the stainless steel.

The delivery device as described is suitable for use in a method for injecting a medication or drug to a patient and preventing or inhibiting an allergic reaction by the stainless steel. The method includes providing a delivery device with a medication compartment and a needle with a distal end configured for piercing the patient where the surface of the needle that contacts the patient is covered by a non-allergenic protective layer. The protective layer can be a coating of a noble metal, such as gold, over the surface of the stainless steel to prevent contact of the stainless steel with the patient.

The above description of the preferred embodiments is not to be deemed as limiting the invention, which is defined by the appended claims. The disclosure is intended to enable the artisan of ordinary skill to practice variants of the invention described without departing from the scope of the invention. Numerical limitations herein, in the specification and in the claims, are understood to be limited by the modifier “about,” such that minor departures yielding equivalent results is within the scope of the invention. Features or dependent claim limitations disclosed in connection with one embodiment or independent claim may be combined in another embodiment or with a different independent claim without departing from the scope of the invention.

1. A medication delivery device comprising:
a dispensing mechanism connected to a reservoir configured for containing a medication; and
a stainless steel needle connected to said dispensing mechanism for delivering the medication, said stainless steel needle having an outer surface configured for contacting the patient, said stainless steel needle having protective layer of a non-allergenic material over at least a portion of said stainless steel needle to prevent direct contact of the stainless steel needle with the patient.

2. The medication delivery device of claim 1, wherein said protective layer is a metal coating of a noble metal.

3. The medication delivery device of claim 1, wherein said protective layer is a metal coating formed directly on said outer surface of said needle, and where said metal coating is selected from the group consisting of gold, platinum, iridium, silver, rhenium, ruthenium, rhodium, palladium, iridium, and osmium.

4. The medication delivery device of claim 1, wherein said protective layer is a gold coating formed directly on said stainless steel needle.

5. The medication delivery device of claim 4, wherein said gold coating is formed on a distal tip of said needle.

6. The medication delivery device according to claim 1, wherein said stainless steel needle has a distal end, a proximal end, and an intermediate surface oriented between said distal end and said proximal end.

7. The medication delivery device according to claim 6, wherein said protective layer is a gold coating formed on said distal end and is spaced distally from said intermediate surface and said spaced from said proximal end.

8. The medication delivery device according to claim 6, wherein said protective layer is a gold coating provided on said intermediate surface and spaced from said distal end and said proximal end.

9. The medication delivery device according to claim 1, wherein said delivery device is a syringe and said stainless steel needle extends from a distal end of said syringe, said protective layer covers an entire length of an exposed portion of said stainless steel needle.

10. The medication delivery device according to claim 1, wherein said delivery device comprises a catheter and said stainless steel is an introducer needle positioned in a lumen of said catheter with a distal end of said stainless steel needle extending from said catheter, and where said distal end of said introducer needle is covered by said protective layer to prevent direct contact of said stainless steel needle with the patient.

11. The medication delivery device of claim 1, wherein said delivery device is a pen needle having a needle hub with a proximal end configured for connecting to a delivery pen, and a distal end supporting said needle.

12. A medication delivery device comprising:
a syringe having a syringe barrel with a proximal end, a distal end, a lumen extending between said proximal end and said distal end, and a stainless steel needle extending from said distal end;
said stainless steel needle having length for injecting a medication into a subject and an outer surface covered by a protective layer of a non-allergenic coating material to prevent contact of an outer surface of the stainless steel needle directly with a tissue of the subject and prevent an allergic reaction in the subject by contact with the stainless steel needle.

13. The medication delivery device according to claim 12, wherein said protective layer is a metal coating of a noble metal.

14. The medication delivery device according to claim 12, wherein said protective layer is a metal coating selected from the group consisting of gold, platinum, iridium, silver, rhenium, ruthenium, rhodium, palladium, iridium, and osmium.

15. The medication delivery device according to claim 12, wherein said protective layer is a gold coating formed directly on an exposed surface of said needle.

16. A method for injecting a medication to a subject and inhibiting an allergic reaction at a delivery site, comprising
providing a delivery device including a medication reservoir, a dispensing mechanism connected to said reservoir, and a stainless steel needle having lumen, a distal end, a proximal end, and an intermediate surface between said distal end and said proximal end, said stainless steel needle having an outer protective layer to prevent direct contact with tissue of a patient; and
contacting said stainless steel needle with the patient and delivering the medication to the patient, said outer protective layer preventing contact of the stainless steel needle with the patient and preventing or inhibiting allergic reactions by the patient from nickel in the stainless steel needle.

17. The method according to claim 16, wherein said outer protective layer is a metal coating selected from the group consisting of gold, platinum, iridium, silver, rhenium, ruthenium, rhodium, palladium, iridium, and osmium.

18. The method according to claim 16, wherein said outer protective layer is gold.

19. The method according to claim 16, wherein said outer protective layer is provided on said distal end of said needle and spaced from said intermediate surface, and where said intermediate surface of said needle is spaced from a surface of said patient when said needle is inserted.

20. The method according to claim 17, wherein said outer protective layer is formed on said intermediate surface, and where said needle is inserted into a lumen where said distal end is oriented in the lumen and the protective layer on the intermediate surface contacts a surface of the patient.
